(19) United States
(12) Patent Application Publication
(10) Pub. No.: US 2024/0325154 A1
(43) Pub. Date: Oct. 3, 2024

(54) IMPLANT WITH OSSEOINTEGRATING STRUCTURE

(71) Applicant: Episurf IP-Management AB, Stockholm (SE)

(72) Inventors: Leif RYD, Malmö (SE); Katarina FLODSTRÖM, Danderyd (SE); Maria WIK, Årsta (SE)

(73) Assignee: Episurf IP-Management AB, Stockholm (SE)

(21) Appl. No.: 18/617,609

(22) Filed: Mar. 26, 2024

(30) Foreign Application Priority Data
Mar. 27, 2023 (SE) 2350345-1

(51) Int. Cl. A61F 2/30; A61B 17/15; A61B 17/17; A61F 2/46

(52) U.S. Cl. CPC A61F 2/30771; A61B 17/15; A61B 17/1775; A61F 2/30749; A61F 2/30942; A61F 2/4606; A61F 2002/30889; A61F 2002/3092; A61F 2002/3093; A61F 2002/4687; A61F 2310/00023; A61F 2310/00029

(57) ABSTRACT

An implant adapted to be attached to an implant receiving surface in a joint of a patient is provided. The implant includes a bone contacting surface, and at least one implant peg extending from the bone contacting surface, wherein both the bone contacting surface and a part of a surface area of the implant peg includes an osseointegrating structure, such as e.g. a lattice structure or a random lattice structure.

### CROSS-REFERENCE TO RELATED APPLICATIONS

This application claims benefit of Swedish Application No. 2350345-1, filed Mar. 27, 2023, which contents are incorporated herein in their entirety by reference.

### TECHNICAL FIELD

The present disclosure relates generally to an implant adapted to be attached to an implant receiving surface in a joint of a patient.

### BACKGROUND

Pain and overuse disorders of the joints of the body is a common problem, that can be caused by for example injury, wear, arthritis, infection, or other cartilage and bone conditions. In a typical anatomical joint, the friction between the cartilage and the surrounding parts of the joint is very low, which facilitates movement of the joint under high pressure. The cartilage is however prone to damage due to disease, injury or chronic wear. Moreover, the cartilage does not readily heal after damages, as opposed to other connective tissue, and if healed, durable hyaline cartilage is often replaced by less durable fibrocartilage. This means that damages of the cartilage have a tendency to gradually become worse. It is therefore important to have efficient means and methods for repairing damaged cartilage and underlying bone, that may have developed into osteoarthritis, in joints.

The advantages of using implants for repairing damaged cartilage and underlying bone have stimulated the development of small joint implants, suitable for repair of injuries to cartilage and/or underlying bone that have a minimal influence on the surrounding parts of the joint. Such small implants are often designed with an implant body that may be formed as a plate with a wear resistant articulating surface for facing the articulate side of the joint, and a bone contacting surface for facing the bone below the damaged part of cartilage. The shape and the curvature of the articulating surface of the implant may be designed to be similar to the shape and the curvature of the part of the joint where the implant is inserted. Such small implants are often designed with a mushroom-like shape, having an implant body, or head, and one or more pegs, rods, or screws, projecting from the bone contacting side of the implant body for fastening the implant to the bone.

U.S. Pat. Nos. 8,655,468 and 8,644,973 describe implants in which the curvature of the articulating surface is designed to correspond to the curvature of a simulated healthy surface at a site of diseased cartilage in a joint of a patient.

Examples of implants provided with a lattice structure are found in the patent publications US2020197183A1, US215297350A1 and AU201503823A1.

Other examples of related art are found in the patent publications US20110266265, US20140172111A1, US20210177604A1, US20110190899A1, US20110071802A1 and US20200253740A1.

### Problems with the Prior Art

It is important that an implant is properly secured to the implant receiving surface in the bone, for patient comfort and proper functioning of the joint. However, there are situations when it is desirable to remove an implant, e.g. in order to replace it with another implant or treatment. It is then important that the implant is not secured to the extent that it cannot be removed from the joint.

Therefore, there is a need for an improved implant adapted to be attached to an implant receiving surface in a joint of a patient.

### SUMMARY

The above described problem is addressed by the claimed implant adapted to be attached to an implant receiving surface in a joint of a patient. The implant preferably comprises a bone contacting surface, designed to correspond to the implant receiving surface, and at least one implant peg extending from the bone contacting surface, wherein both the bone contacting surface and a part of a surface area of the implant peg comprises an osseointegrating structure, such as e.g. a lattice structure or a random lattice structure. This enables the implant to be properly secured to the implant receiving surface in the bone, while still not being secured to the extent that it cannot be removed from the joint. In case of implant removal, it is desired that the osseointegration is limited to the area closest to the bone contacting surface pf the implant, to minimize unnecessary removal of bone, attached to the implant, from the joint. This is in embodiments achieved by providing such an implant wherein the surface area of the implant peg comprises said osseointegrating structure along less than half of the length of the implant peg.

In embodiments, the surface area of the implant peg comprises the osseointegrating structure along around one third of its length.

In embodiments, the osseointegrating structure has a depth in the range of 0.2-1.0 mm, for example a depth of 0.5 mm.

In embodiments, the osseointegrating structure has a cell size in the range of 50-1500 micron, for example a cell size in the range of 100-1000 micron.

In embodiments, the osseointegrating structure has a volume porosity in the range of 20-80%, for example a volume porosity in the range of 30-70%.

In embodiments, the implant peg has a first diameter along most of its length, and a smaller diameter at the end, either with an edge where the first diameter suddenly changes into a second, smaller, diameter, or by the implant peg being tapered towards its end. The surface area of the implant peg then preferably comprises the osseointegrating structure along less than two thirds of the length of the implant peg having the first diameter, e.g. along around half of the length having the first diameter.

In embodiments, an articulating surface of the implant is designed to correspond to the curvature of a simulated healthy articulating surface at a site of diseased cartilage and/or bone, where the contour curvature of the articulating surface is generated based on a determined surface curvature of the cartilage and/or the bone in a predetermined area at the site of diseased cartilage and/or bone, to mimic the original, undamaged, articulating surface of the joint.

In embodiments, the articulating surface of the implant comprises a positioning mark.

In embodiments, the articulating surface of the implant comprises titanium (Ti) or titanium alloy, titanium nitride (TiN) titanium niobium nitride (TiNbN), and/or a cobalt-chromium (CoCr) alloy.

In embodiments, the implant is manufactured by additive manufacturing. This is a simple way of creating the osseointegrating structure on the implant.

The above described problem is further addressed by the claimed surgical kit, comprising the above implant and a bone processing tool guide comprising a contact surface configured to have a shape and contour that is designed to correspond to and to fit the actual surface contour in a predetermined area of a joint of a patient.

In embodiments, the surgical kit further comprises an insert tool configured to be used for attaching the implant to the implant receiving surface, wherein the insert tool has an implant engaging portion that has a surface curvature that substantially corresponds to the surface curvature of the articulating surface of the implant.

The above described problem is also addressed by the claimed system for customizing an implant adapted to be attached to an implant receiving surface in a joint of a patient. The system preferably comprises at least one processor configured to: obtain a three-dimensional image representation of at least a part of the joint of the patient based on medical images generated using a medical imaging system; determine the shape and dimensions of a customized implant adapted to be attached to the implant receiving surface, by designing a bone contacting surface of the implant to correspond to the implant receiving surface and designing the implant to comprise at least one implant peg extending from the bone contacting surface; and add an osseointegrating structure, such as e.g. a lattice structure or a random lattice structure, to the bone contacting surface of the implant and to a surface area of the implant peg along less than half of the length of said implant peg.

The above described problem is further addressed by the claimed method for customizing an implant adapted to be attached to an implant receiving surface in a joint of a patient. The method preferably comprises: obtaining a three-dimensional image representation of at least a part of the joint of the patient based on medical images generated using a medical imaging system; determining the shape and dimensions of a customized implant adapted to be attached to the implant receiving surface, by designing a bone contacting surface of the implant to correspond to the implant receiving surface and designing the implant to comprise at least one implant peg extending from the bone contacting surface; and adding an osseointegrating structure, such as e.g. a lattice structure or a random lattice structure, to the bone contacting surface of the implant and to a part of a surface area of the implant peg along less than half of the length of said implant peg.

In embodiments, an osseointegrating structure is added along less than half of the length of the implant peg, e.g. along around one third of its length.

In embodiments, an osseointegrating structure is added in one or more of the following:
in the form of a lattice structure with a depth in the range of 0.2-1.0 mm, for example a depth of 0.5 mm; and/or
in the form of a lattice structure with a cell size in the range of 50-1500 micron, for example a cell size in the range of 100-1000 micron;
   and/or
in the form of a lattice structure with a volume porosity in the range of 20-80%, for example a volume porosity in the range of 30-70%.

In embodiments, the implant peg is designed to have a first diameter along most of its length, and a smaller diameter at the end, either with an edge where the first diameter suddenly changes into a second, smaller, diameter, or by the implant peg being tapered towards its end. The osseointegrating structure is then preferably added along less than two thirds of the length of the implant peg having the first diameter, e.g. along around half of the length having the first diameter.

In embodiments, the contour curvature of the articulating surface is generated using said three-dimensional image representation of the joint, to be based on the determined surface curvature of the cartilage and/or the bone in a predetermined area of the joint, to mimic the original, undamaged, articulating surface.

In embodiments, the generation of the contour curvature of the articulating surface comprises simulating a healthy articulating surface in the predetermined area, and designing the surface of the customized implant to match said simulated healthy articulating surface.

The anatomical joint may be e.g. a knee, an ankle, a hip, a toe, an elbow, a shoulder, a finger, or a wrist.

The term “osseointegrating structure” in this disclosure comprises any shape of the surface structure that enhances osseointegration. A lattice structure or a random lattice structure are given as examples of osseointegrating structures, but other types of uneven surface structures are also encompassed by the term. The surface structure may e.g. have a grid shape, or an entirely random shape, as long as there are a number of different levels of recesses, regular or irregular, in the surface structure.

The term “peg” in this disclosure comprises also pegs in the shape of screws. The end of the implant peg is defined as the end directed away from the bone contacting surface of the implant peg.

The medical imaging system may e.g. be a magnetic resonance imaging (MRI) system, an x-ray imaging system, an ultrasonic imaging system, a fluoroscopic imaging system and/or a computer tomography (CT) system, e.g. CBCT. The medical images may be a number of images in a series captured during a process of scanning through different layers of the anatomical joint or part of it using a medical imaging system.

The processor may in some embodiments comprise several different processors which together perform the claimed functions.

The scope of the invention is defined by the claims, which are incorporated into this section by reference. A more complete understanding of embodiments of the invention will be afforded to those skilled in the art, as well as a realization of additional advantages thereof, by a consideration of the following detailed description of one or more embodiments. Reference will be made to the appended sheets of drawings that will first be described briefly.

### BRIEF DESCRIPTION OF THE DRAWINGS

FIG. 1 shows a schematic view of a system for customizing an implant, in accordance with one or more embodiments described herein.

FIGS. 2a-b illustrate a metatarsophalangeal implant arrangement for repairing damage in a metatarsophalangeal joint, in accordance with one or more embodiments described herein.

FIGS. 3a-b illustrate embodiments of a metatarsal implant, in accordance with one or more embodiments described herein.

FIGS. 4a-b illustrate the creation of an implant receiving surface suitable for receiving a metatarsal implant on a first metatarsal head, in accordance with one or more embodiments described herein.

FIG. 4c illustrates a first metatarsal bone comprising an implant receiving surface suitable for receiving a metatarsal implant on a first metatarsal head, in accordance with one or more embodiments described herein.

FIGS. 4d-e illustrate embodiments of a metatarsal implant positioned on a first metatarsal head, in accordance with one or more embodiments described herein.

FIGS. 5a-h illustrate embodiments of a metatarsal saw guide, in accordance with one or more embodiments described herein.

FIG. 6a illustrates a metatarsal implant dummy, in accordance with one or more embodiments described herein.

FIG. 6b illustrates a metatarsal drill guide, in accordance with one or more embodiments described herein.

FIG. 7 is a schematic flow diagram for a method for customizing an implant, in accordance with one or more embodiments described herein.

Embodiments of the present disclosure and their advantages are best understood by referring to the detailed description that follows. It should be appreciated that like reference numerals are used to identify like elements illustrated in one or more of the figures.

### DETAILED DESCRIPTION

### Introduction

It is important that an implant is properly secured to the implant receiving surface in the bone, for patient comfort and proper functioning of the joint. However, there are situations when it is desirable to remove an implant, e.g. in order to replace it with another implant or treatment. It is then important that the implant is not secured to the extent that it cannot be removed from the joint.

The present disclosure relates generally to an implant adapted to be attached to an implant receiving surface in a joint of a patient. Embodiments of the disclosed solution are presented in more detail in connection with the figures.

### System Architecture

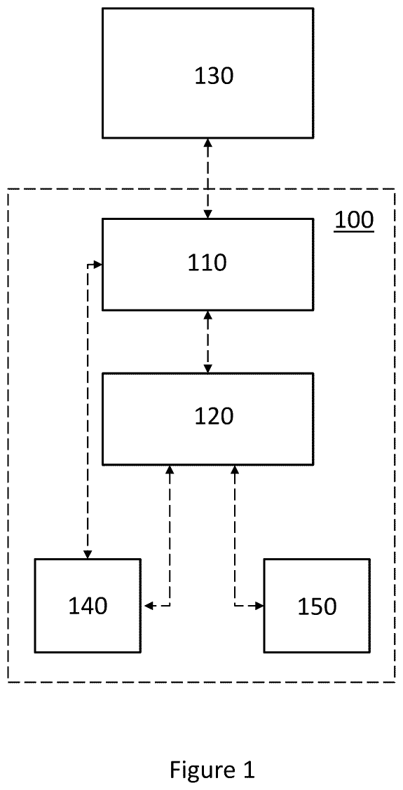

FIG. 1 shows a schematic view of a system 100 for customizing an implant suitable for use in a joint of a patient. The implant is adapted to be attached to an implant receiving surface which has been created in the joint of a patient. According to embodiments, the system 100 comprises a display 140, at least one manipulation tool 150, and a storage media 110, configured to receive and store image data and parameters. In some embodiments, the system 100 is communicatively coupled to a medical imaging system 130. The medical imaging system 130 may be configured to capture or generate medical images, e.g. radiology images such as X-ray images, ultrasound images, computed tomography (CT), e.g. CBCT, images, nuclear medicine including positron emission tomography (PET) images, and magnetic resonance imaging (MRI) images. The storage media 110 may be configured to receive and store medical images from the medical imaging system 130. In embodiments, medical images are uploaded into the storage media 110 by personnel at a medical care facility, preferably the medical care facility where the medical imaging takes place. Medical images may however also be uploaded into the storage media 110 by another medical care facility, or by other authorized personnel. The uploading of the medical images may also be an automatic uploading directly from one system to another.

In one or more embodiments, the system 100 comprises at least one processor 120 configured to: obtain a three-dimensional image representation of a joint of a patient based on medical images generated using a medical imaging system 130; determine damage to the joint of the patient by analyzing medical images generated using a medical imaging system 130; and determine the shape and dimensions of a customized implant 300 suitable for repairing said determined damage, using said three-dimensional image representation of the joint, wherein the contour curvature of the articulating surface 310 is generated based on the determined surface curvature of the cartilage and/or the bone in a predetermined area at the site of diseased cartilage and/or bone, to mimic the original, undamaged, articulating surface or to a selected articulate surface contour.

In one or more embodiments, the at least one processor 120 is configured to determine the shape and dimensions of the customized implant 300 by simulating a healthy articulating surface at the site of the determined damage, including designing the surface of the customized implant 300 to match the simulated healthy articulating surface. The determination of the shape and dimensions of a customized implant 300 preferably involves designing an implant surface that corresponds to a 3D image of a simulated healthy cartilage surface or of a selected articulate surface contour.

In embodiments, the at least one processor 120 is configured to also output the shape and dimensions of the customized implant 300 as parameters for manufacturing said customized implant 300.

The at least one processor 120 may for example be a general data processor, or other circuit or integrated circuit capable of executing instructions to perform various processing operations. The at least one processor 120 may in some embodiments comprise several different processors 120 which together perform the claimed functions. In the same way, the storage media 110 may in some embodiments comprise several different storage media 110 which together perform the claimed functions.

The display 140 may be configured to receive image data for display via the processor 120, and/or to retrieve image data for display directly from the storage media 110, possibly in response to a control signal received from the processor 120 or the at least one manipulation tool 150.

The processor 120 may further be configured to perform any or all of the method steps of any or all of the embodiments presented herein.

The claimed invention is herein exemplified by embodiments of a metatarsophalangeal implant arrangement, but as mentioned above the inventive solution is in embodiments applied in implants configured for any anatomical joint may be e.g. a knee, an ankle, a hip, a toe, an elbow, a shoulder, a finger, or a wrist. The implants generally comprises an implant body with an articulating surface and a bone contacting surface, and a peg extending from the bone contacting surface.

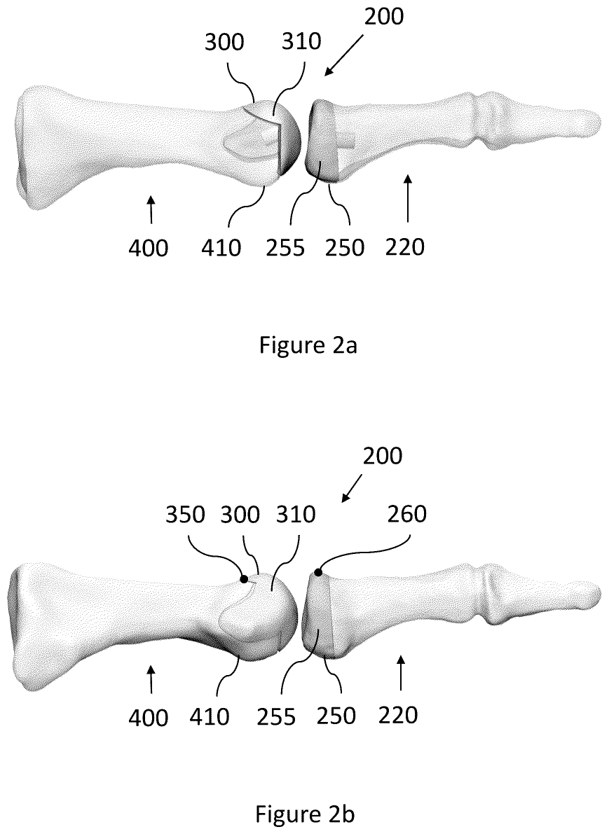

FIGS. 2a-b illustrate a metatarsophalangeal implant arrangement 200 for repairing damage in a first metatarsophalangeal joint of a patient, comprising a metatarsal implant 300 and a phalangeal implant 250. The implants 250, 300 are illustrated as implanted into the first metatarsophalangeal joint, with the metatarsal implant 300 attached to the first metatarsal head 410 and the phalangeal implant 250 attached to the proximal end of the first proximal phalanges 220. The implants 250, 300 comprise articulating surfaces 255, 310 that are designed to allow them to interact with each other when the implants 250, 300 are implanted into the first metatarsophalangeal joint of the patient.

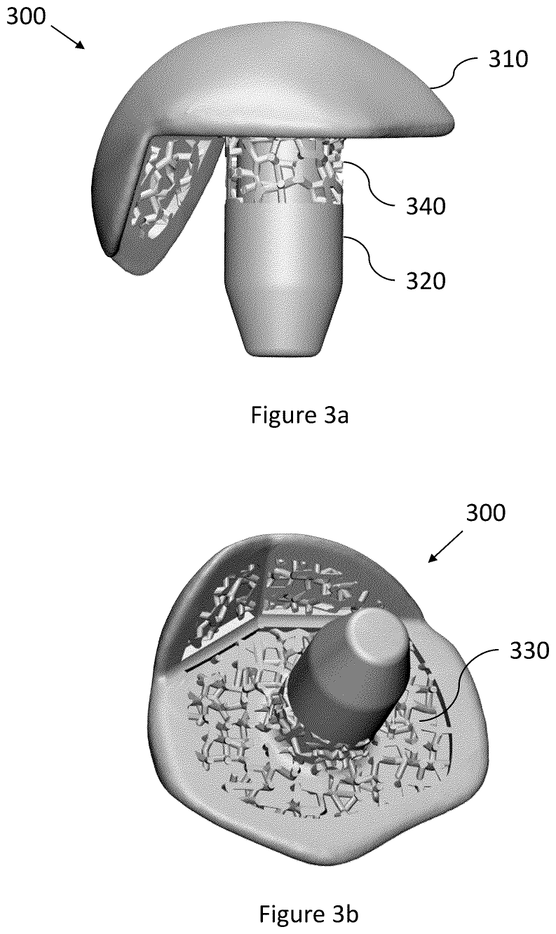

FIGS. 3a-b illustrate a metatarsal implant 300 for repairing damage in a first metatarsophalangeal joint of a patient. The metatarsal implant 300 is preferably configured to be attached to an implant receiving surface 420 which has been created on a metatarsal head 410 by sawing off sections of the metatarsal head 410. Since there is often damage on more than one side of the metatarsal head 410, it is often desirable for the metatarsal implant 300 to be shaped so that it encloses the end of the metatarsal head 410. There may e.g. be osteophytes that need to be removed from the metatarsal head 410, and in that case the metatarsal implant 300 preferably encloses the portions of the metatarsal head 410 where such osteophytes have been removed. The metatarsal implant 300 preferably comprises an implant peg 320 extending from a bone contacting surface 330.

The metatarsal implant 300 may be manufactured in a number of different ways, including 3D printing. The articulating surface 310 of the metatarsal implant 300 is preferably a metal, metal alloy or ceramic surface, e.g. comprising titanium (Ti), titanium alloy, titanium nitride (TiN), titanium niobium nitride (TiNbN), and/or a cobalt-chromium (CoCr) alloy. It is preferably polished to a perfectly smooth surface, with a very low surface roughness, to lower the risk of a sesamoid bone locking against the articulating surface 310 of the metatarsal implant 300. The bone contacting surface 330 of the metatarsal implant 300 may be coated with an osseointegrating and/or bioactive material, such as e.g. hydroxyapatite. The bone contacting surface 330 of the metatarsal implant 300 may alternatively be coated with titanium (Ti), titanium alloy, titanium nitride (TiN), or titanium niobium nitride (TiNbN), This reduces the need for using an adhesive for securing the metatarsal implant 300 to the metatarsal head 410, but an adhesive (such as e.g. bone cement) may be used anyhow.

As illustrated in FIG. 3a-b, the bone contacting surface 330 of the metatarsal implant 300 comprises an osseointegrating structure 340, such as e.g. a lattice structure or a random lattice structure, in order to improve osseointegration. The osseointegrating structure may comprise any shape of the surface structure that enhances osseointegration, specifically different types of uneven surface structures. The surface structure may e.g. have a grid shape, or an entirely random shape, as long as there are a number of different levels of recesses, regular or irregular, in the surface structure.

Such an osseointegrating structure 340 preferably extends also down onto the implant peg 320. In preferred embodiments, at least a part of a surface area of the implant peg 320 comprises such an osseointegrating structure 340. However, it is preferred that the osseointegrating structure 340 extends along less than half of the length of the implant peg 320, e.g. along around a third of its length, since it may otherwise be too difficult to remove the metatarsal implant 300 at a later stage. The surface area of the implant peg 320 may also be coated with an osseointegrating and/or bioactive material, such as e.g. hydroxyapatite. The surface area of the implant peg 320 may alternatively be coated with titanium (Ti), titanium alloy, titanium nitride (TiN), or titanium niobium nitride (TiNbN).

The implant peg 320 is preferably designed for press-fit into a recess made in the bone. The implant peg 320 may have a smaller diameter at the end, for easier insertion into the recess. If an adhesive such as e.g. bone cement is used, it may not be necessary for the implant peg 320 to be designed for press-fit into the recess. The use of press-fit (where the implant peg is slightly larger than the recess) secures the implant 300 to the implant receiving surface 420 on the metatarsal head 410 regardless of whether an adhesive such as bone cement is used, but the combination of press-fit and adhesive of course secures the implant 250, 300 even more to the implant receiving surface 420. The implants 300 may comprise one or more recesses for bone cement in the implant peg 320, which secures the implant 300 even further. The implant peg 320 may have the shape of a screw.

The metatarsal implant 300 illustrated in FIGS. 3a-b comprises an implant peg 320 which is tapered at the end, and thus has a first diameter along most of its length, and is tapering towards a second, smaller diameter at the end. Instead of being tapered at the end, the implant peg 320 may however have an edge where the first diameter suddenly changes into a second, smaller, diameter. In such embodiments, the surface area of the implant peg 320 preferably comprises the osseointegrating structure 340 along less than two thirds of the length of the implant peg 320 having the first diameter, e.g. along around half of the length having the first diameter.

The metatarsal implant 300 may also comprise a positioning mark 350, preferably positioned on the articulating surface 310. This makes it easier to accomplish a correct rotational positioning of the implant during surgery, which may be important because the articulating surface of the implant will in most situations not be rotationally symmetric. The positioning mark 350 may e.g. be a rotational positioning mark, or an indication of a direction in relation to the anatomy of the joint.

The surface curvature of the articulating surface 310 of the metatarsal implant 300 preferably corresponds as closely as possible to the surface curvature of the undamaged metatarsal head 410. In this way, full account is taken of the sesamoid bones, which move around the metatarsal head as the toe is bent. This increases the possibility of the patient being given back a full range of motion.

By analyzing the surface curvature of the cartilage and/or the bone in a predetermined area comprising and surrounding the site of diseased cartilage, it is possible to simulate a healthy articulating surface of the damaged metatarsal head 410 and mimic the original, undamaged, articulating surface of the metatarsal head 410. The image data may be analyzed in a data processing system to identify and determine physical parameters for the cartilage and/or bone damage. The physical parameters to be determined may comprise the presence, the location and the size and shape of the cartilage and/or bone damage, as well as curvature of the surface contour of the cartilage or the bone in an area of the cartilage and/or bone damage.

When such a healthy articulating metatarsal surface has been simulated, it is possible to design an individualized metatarsal implant 300 with an articulating surface 310 that corresponds to the simulated healthy metatarsal surface.

However, it is also possible to select the best matching predefined surface from a limited number of different predefined surfaces. This enables the use of standardized metatarsal implants 300. In this way, a set of standardized metatarsal implants 300 of different dimensions may be manufactured and stored, to be later used for repairing damage in the metatarsophalangeal joint.

A standardized metatarsal implant 300 may in this case be selected from a predefined set of standardized metatarsal implants 300 having varying dimensions. The predefined set of standardized metatarsal implants 300 is preferably created by analyzing dimensional data from stored images of the metatarsal head 410 from a large number of different patients. The standardized metatarsal implant 300 should be selected as a standardized metatarsal implant 300 having dimensions that match the shape of the metatarsal head 410 of the patient, thereby making it suitable for repairing the determined damage. A 3D model of the metatarsophalangeal joint, visualizing the determined damage, may be used in order to determine which standardized metatarsal implant 300 is the best fit for the metatarsal head 410 of the patient.

However, even if it is possible to use a standardized metatarsal implant 300, there will always be cases where it cannot be ascertained that a standardized metatarsal implant 300 will really fit the implant receiving surface 420 on the metatarsal head 410, and repair the damage while taking full account of the sesamoid bones. In order to ascertain that the metatarsal implant 300 will really fit the implant receiving surface 420 on the metatarsal head 410, and repair the damage while taking full account of the sesamoid bones, it is necessary to design an individualized metatarsal implant 300 with an articulating surface 310 that corresponds to the simulated healthy metatarsal surface, which may also extend far enough to always interact with the sesamoid bones.

The metatarsal implant 300 may be used alone, or together with a phalangeal implant 250 in a metatarsophalangeal implant arrangement 200, as illustrated in FIGS. 2a-b. In such a metatarsophalangeal implant arrangement 200, it is preferred if either both the metatarsal implant 300 and the phalangeal implant 250 are individualized, or both the metatarsal implant 300 and the phalangeal implant 250 are standardized. However, it is also possible to combine an individualized metatarsal implant 300 with a standardized phalangeal implant 250, or a standardized metatarsal implant 300 with an individualized phalangeal implant 250.

FIGS. 3a-b specifically illustrate a metatarsal implant 300, but the discussion in this disclosure applies to any kind of implant 300 suitable for use in a joint of a patient. The present disclosure thus relates to any kind of implant 300 adapted to be attached to an implant receiving surface 420 in a joint of a patient, the implant 300 comprising a bone contacting surface 330, designed to correspond to the implant receiving surface 420, and at least one implant peg 320 extending from the bone contacting surface 330. In such an implant 300, preferably both the bone contacting surface 330 and at least a part of a surface area of the implant peg 320 comprises an osseointegrating structure 340, such as e.g. a lattice structure or a random lattice structure. The surface area of the implant peg 320 preferably comprises the osseointegrating structure 340 along less than half of the length of the implant peg 320, for example as in embodiments around along one third of the length of the implant peg. If the implant peg 320 has a first diameter along most of its length, and a smaller diameter at the end (either with an edge where the first diameter suddenly changes into a second, smaller, diameter, or by the implant peg 320 being tapered towards its end), the surface area of the implant peg 320 preferably comprises the osseointegrating structure 340 along less than two thirds of the implant peg 320 having the first diameter, e.g. along around half of the length having the first diameter.

In embodiments, the implant 300 is manufactured by additive manufacturing. This is a simple way of creating the osseointegrating structure on the implant 300.

It has been found that an advantageous balance between reliable fixation of an implant to a bone is achieved by providing both the bone contacting surface and a limited part of a peg extending from the bone contacting surface with an osseointegrating structure. With an osseointegrating structure along less than a half of the length of the implant peg, and in embodiments along around a third of the implant peg, it is enabled to remove the implant when needed without destroying an unnecessary amount of bone structure. Preferably, the osseointegrating structure is placed in the part of the peg closest to the bone contacting surface, but may in embodiments be placed elsewhere along the length of the peg.

The osseointegrating structure is in embodiments in the form of a lattice structure. Such lattice structures may in different embodiments be regular or randomized.

Advantageous adhesive effect has been found in embodiments wherein:
the osseointegrating structure has a depth in the range of 0.2-1.0 mm, for example a depth of 0.5 mm;
   and/or
the osseointegrating structure has a cell size in the range of 50-1500 micron, for example a cell size in the range of 100-1000 micron;
   and/or
the osseointegrating structure has a volume porosity in the range of 20-80%, for example a volume porosity in the range of 30-70%.

One or more of these properties are employed in embodiments of implant, surgical kit, system and method, as described herein.

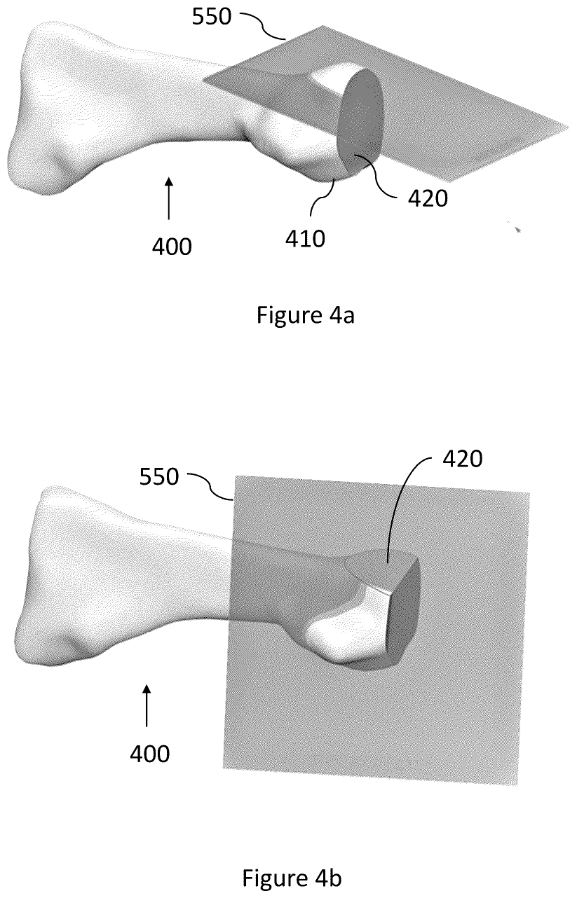

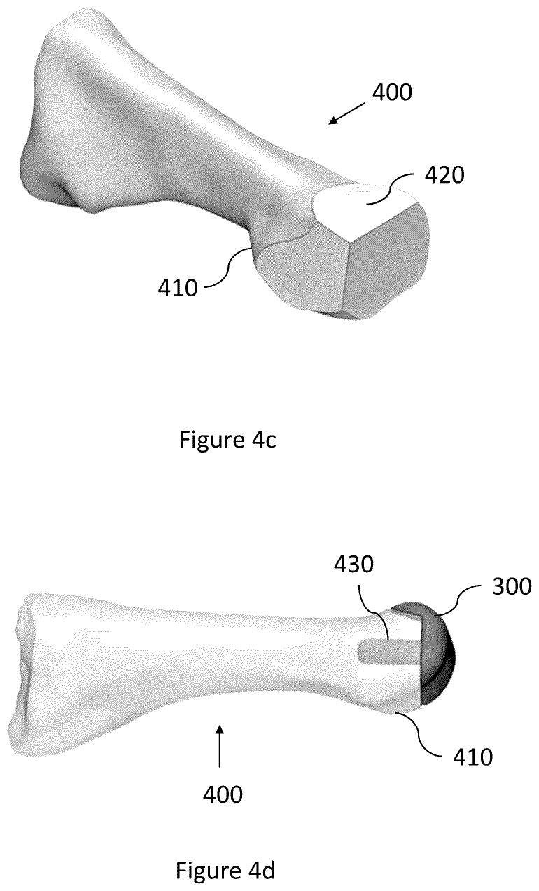

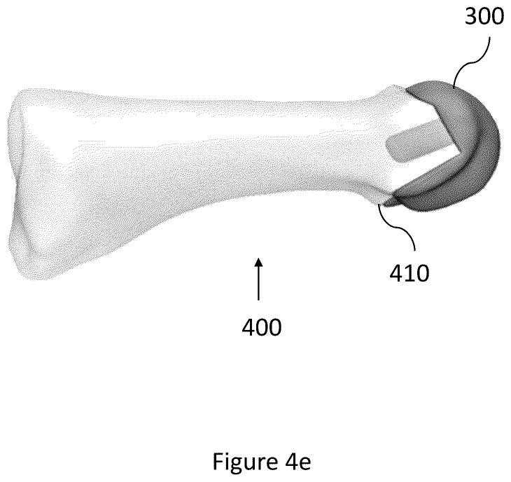

FIGS. 4a-b illustrate the creation of an implant receiving surface 420 suitable for receiving a metatarsal implant 300 on a metatarsal head 410, FIG. 4c illustrates a first metatarsal bone 400 comprising an implant receiving surface 420 suitable for receiving a metatarsal implant 300 on a first metatarsal head 410, and FIG. 4d-e illustrate embodiments of a metatarsal implant 300 positioned on a first metatarsal head 410.

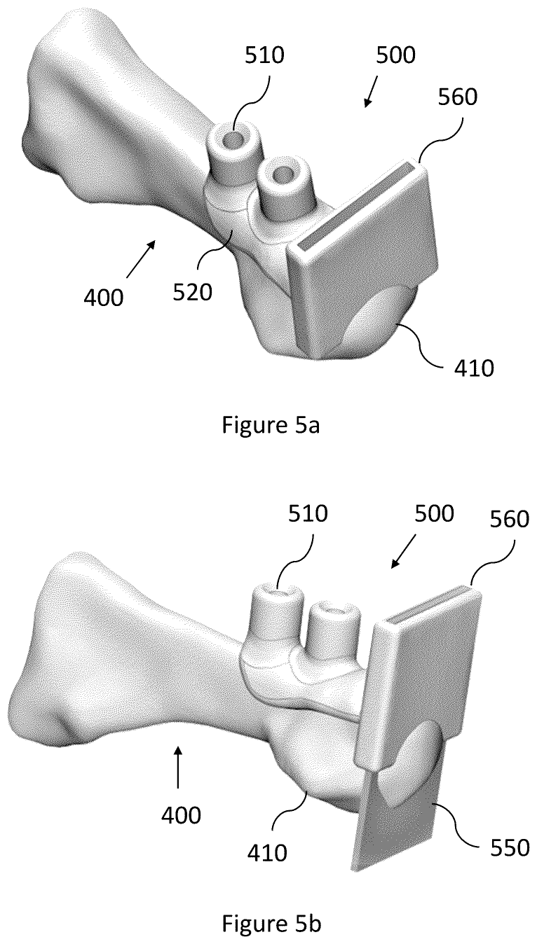

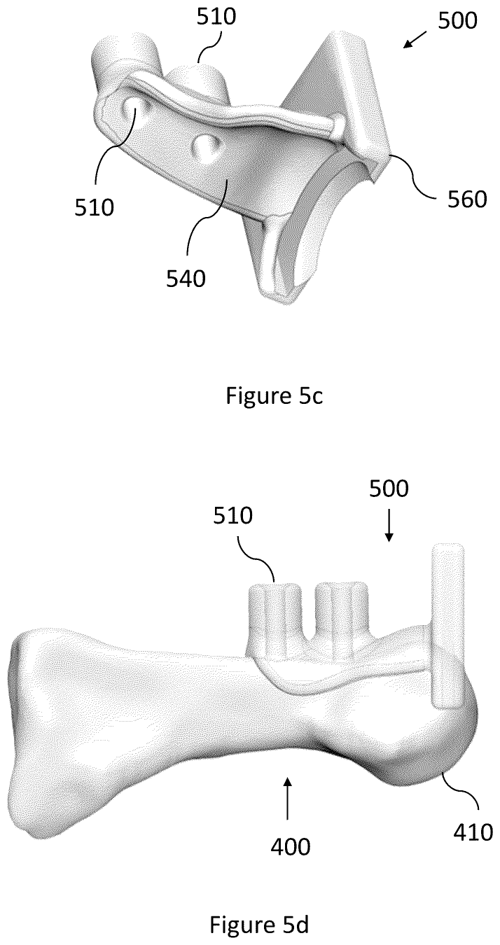

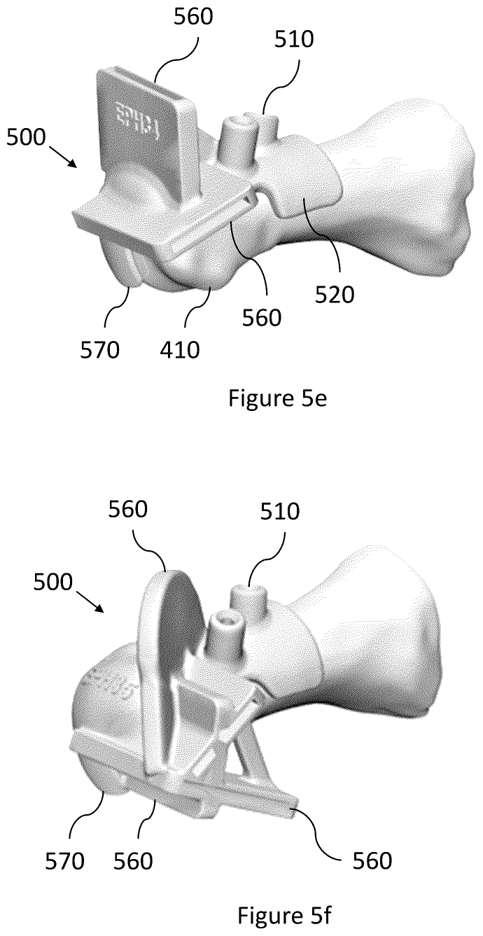

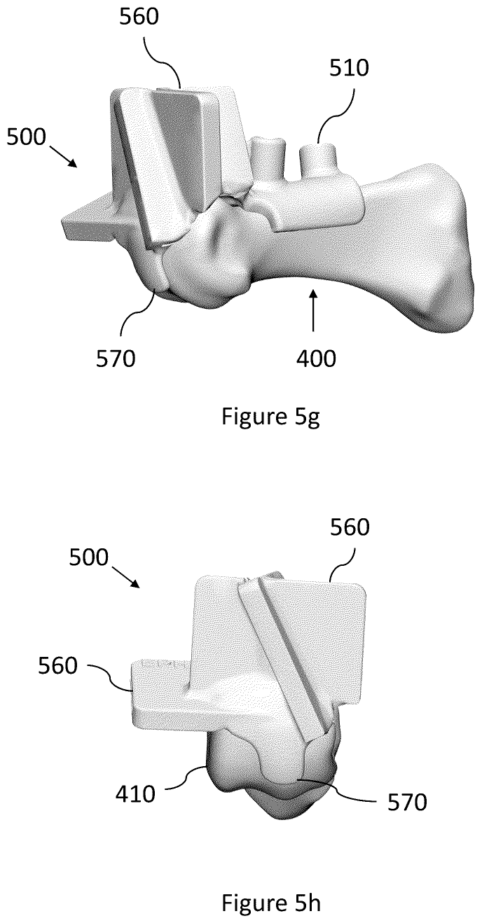

FIGS. 5a-h illustrate embodiments of a bone processing tool in the form of a metatarsal saw guide for creating an implant receiving surface 420 on a metatarsal head 410. The saw guide 500 preferably has a contact surface 540 that has a shape and contour that is designed to correspond to and to fit the actual contour of the metatarsal bone 400 in a predetermined area of the metatarsal bone 400. The predetermined area is preferably located to allow the creation of an implant receiving surface 420 at the site of diseased cartilage. Thereby, the contact surface 540 of the saw guide 500 corresponds to and fits to the actual contour of the surface of the metatarsal bone 400 at the selected location. The whole contact surface 540 does not have to correspond to the actual contour curvature of the metatarsal surface at the selected location, it is enough if the contact surface 540 comprises at least three contact points, so that the metatarsal saw guide 500 will have a stable mounting in the correct position at the selected location. This helps ensuring that the implant receiving surface 420 will be created in the exact position of the determined damage. Such contact points are preferably chosen to provide maximum support and positional stability for the metatarsal saw guide 500.

The contact surface 540 of the metatarsal saw guide 500 may be stabilized by comprising one or more contact surface extensions 570 that extend around at least a part of the metatarsal head 410, as illustrated in FIGS. 5e-h. Such contact surface extensions 570 help ensuring that the metatarsal saw guide 500 is positioned in the exact desired position on the metatarsal bone 400, and also increases the stability of the metatarsal saw guide 500 on the metatarsal bone 400. Since the metatarsal bone 400 is quite a small bone, exact positioning and stability of the metatarsal saw guide 500 is more important than for larger bones. The contact surface 540 of the metatarsal saw guide 500 may be further stabilized by being attached to the metatarsal surface 400 with one or more nails, rivets, wires or similar attachment means that are inserted into through-holes 510 in the metatarsal saw guide 500. Such additional attachment gives additional support and stability, and enables the contact surface 540 of the metatarsal saw guide 500 to be as small as possible, which is especially important for the metatarsal bone 400, since it is quite small.

The contact surface of the metatarsal saw guide 500 mainly contacts the metatarsal bone, so it may also be called a bone contact surface.

The metatarsal saw guide 500 illustrated in FIGS. 5a-d is arranged to receive a saw blade 550 for sawing off the end of the metatarsal head 410. The metatarsal saw guide 500 illustrated in FIGS. 5e-h is arranged to receive saw blades 550 in different positions for sawing off different sections of the metatarsal head 410, in order to create an asymmetrical implant receiving surface 420, which locks the metatarsal implant 300 in a position where it cannot be rotated. The metatarsal saw guide 500 is preferably arranged so that the same base section 520 can receive different saw blade guides 560, e.g. by the saw blade guides 560 being clicked onto the base section 520, without the base section 520 having to be removed from the metatarsal bone 400. In the embodiment illustrated in FIGS. 5e-h, the metatarsal saw guide 500 is instead a “multiguide”, that comprises a number of different saw blade guides 560. The same metatarsal saw guide 500 could thus be used for receiving saw blade guides 560 for the saw blades 550 for sawing on all desired sides of the metatarsal head 410, in order to create an asymmetrical implant receiving surface 420, which locks the metatarsal implant 300 in a position where it cannot be rotated. Alternatively, a number of different metatarsal saw guides 500 may be used. It is then preferred if the same nails, rivets, wires or similar attachment means are used for all the different metatarsal saw guides 500, so that unnecessary drilling of holes in the metatarsal bone 400 can be avoided.

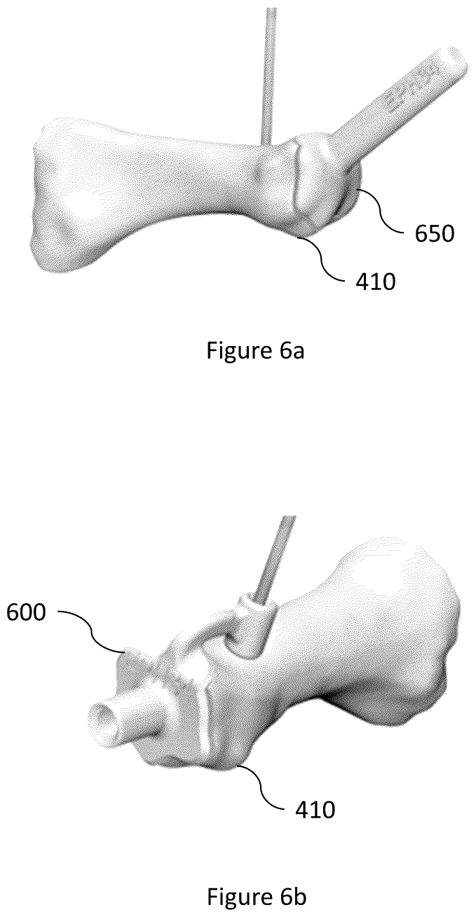

When the implant receiving surface 420 has been created on the metatarsal head 410 using the saw guide 500, a metatarsal implant dummy 650 (as illustrated in FIG. 6a) is preferably used for verifying that the implant receiving surface 420 has the correct size and shape for receiving the metatarsal implant 300, before the recess for the implant peg 320 is drilled using another bone processing tool guide in the form of a metatarsal drill guide 600 (illustrated in FIG. 6b). The metatarsal drill guide 600 preferably comprises a contact surface 640 configured to have a shape and contour that is designed to correspond to and to fit the implant receiving surface 420 on the metatarsal head 410. The metatarsal drill guide 600 is also preferably arranged to use the same nails, rivets, wires or similar attachment means as was used for the metatarsal saw guide 500, so that unnecessary drilling of holes in the metatarsal bone 400 can be avoided.

The metatarsal drill guide 600 may be arranged to have the shape of the implant dummy, so that the metatarsal drill guide 600 may be used for verifying that the implant receiving surface 420 has the correct size and shape for receiving the metatarsal implant 300, before the recess for the implant peg 320 is drilled. In this case, no metatarsal implant dummy 650 is necessary.

The metatarsal saw guide 500 and/or the metatarsal drill guide 600 may comprise a rotational position indicator, which may be used to make a marking on the cartilage or bone at the side of the implant receiving surface 420 on the metatarsal head 410. Such a marking may then be used to correctly rotate the metatarsal implant 300 before the metatarsal implant 300 is positioned on the implant receiving surface 420 on the metatarsal head 410. If the metatarsal implant 300 comprises a positioning mark 350, the alignment of this positioning mark 350 with the marking at the side of the implant receiving surface 420 on the metatarsal head 410 ensures that the metatarsal implant 300 is correctly positioned on the implant receiving surface 420. The metatarsal saw guide 500 and/or the metatarsal drill guide 600 is then preferably configured to allow such a marking to be made while the metatarsal saw guide 500 and/or the metatarsal drill guide 600 is attached to the metatarsal bone 400. The metatarsal saw guide 500 and/or the metatarsal drill guide 600 may for this purpose comprise an indentation at the position of the rotational position indicator on the metatarsal saw guide 500 and/or the metatarsal drill guide 600. The marking may e.g. be added to the cartilage surface by inserting a marking pen into the indentation in the metatarsal saw guide 500 and/or the metatarsal drill guide 600 when the metatarsal saw guide 500 and/or the metatarsal drill guide 600 is attached to the metatarsal bone 400. However, the metatarsal implant 300 may also comprise a positioning mark 350 that is simply indication of a direction in relation to the anatomy of the joint. In this case, no marking on the cartilage or bone is needed.

A correct rotational positioning of the metatarsal implant 300 is important because the articulating surface 310 of the metatarsal implant 300 will in most situations not be rotationally symmetric. An important reason for designing the articulating surface 310 of the metatarsal implant 300 to match the simulated healthy articulating surface of the metatarsal head 410 is to ensure that the metatarsal implant 300 fits smoothly on the metatarsal head 410 and takes full account of the sesamoid bones. If the metatarsal implant 300 is not attached in the correct rotational position, there is a risk that the sesamoid bones will lock against the metatarsal implant 300, and that the patient will thus not be given back a full range of motion.

A marking on the cartilage surface makes it easy for the surgeon to attach the metatarsal implant 300 to the metatarsal head 410 with a correct rotational positioning, if the metatarsal implant 300 also comprises a positioning mark 350. However, the metatarsal implant 300 may also comprise a positioning mark 350 that is simply indication of a direction in relation to the anatomy of the joint.

Insert tools may be used to aid the positioning of the metatarsal implant 300 on the implant receiving surface 420 of the metatarsal head 410. It is e.g. possible to use a mandrel as an insert tool, as is commonly known for trochlear implants. There may be a positioning mark on the insert tool, so that the implant engaging portion may be correctly rotated with respect to the metatarsal implant 300.

In order to repair the damaged cartilage in the metatarsal head 410, a metatarsal surgical kit comprising the above described metatarsal implant 300, the above described at least one metatarsal saw guide 500, the above described metatarsal drill guide 600, and potentially also an insert tool may be used. Even if the metatarsal implant 300 is an implant selected from a predefined set of standardized implants having varying dimensions, it is still preferred to use a customized metatarsal saw guide 500, having a contact surface 540 configured to have a shape and contour that is designed to correspond to and to fit the actual contour of the surface of the metatarsal bone 400 in a predetermined area of the metatarsal bone 400, since this will ensure that the metatarsal saw guide 500 will have a stable mounting in the correct position on the metatarsal bone 400. This helps ensuring that the implant receiving surface 420 will be created in the exact position of the determined damage. However, the metatarsal drill guide 600 may be standardized, since its contact surface 640 need only be adapted to the implant receiving surface 420, which for standardized implants is preferably standardized.

When there is damage in both the proximal phalanges 220 and the metatarsal head 410, a metatarsophalangeal surgical kit may instead be used. The metatarsophalangeal surgical kit may comprise the above described metatarsophalangeal implant arrangement 200, the above described at least one metatarsal saw guide 500, a phalangeal guide tool for the phalangeal implant 250, and one or more insert tools. The metatarsal saw guide 500 for the metatarsal implant and/or the phalangeal guide tool for the phalangeal implant 250 preferably comprise visual markings, so that they are visually distinct from each other. In this way, it will be clear to the surgeon which guide tool to use for which implant. The phalangeal guide tool may be similar to the above described metatarsal saw guide 500, or it may instead be a drill guide, depending on the shape of the phalangeal implant 250.

The insert tools may be one or more mandrels to aid positioning of the implants 250, 300. A surgical kit may also comprise further instruments, such as e.g. an implant dummy 650 (as illustrated in FIG. 6a) for verifying that the implant receiving surface 420 has the correct size and shape for receiving the metatarsal implant 300 before attachment of the implant. For a metatarsophalangeal surgical kit, it is advantageous if either all the instruments associated with the phalangeal implant 250 or all the instruments associated with the metatarsal implant 300 comprise visual markings, so that it is visually clear which instruments belong together. The visual markings may be any type of markings that are visible to a surgeon, such as e.g. color markings. However, the visual markings may also be tactile, such as ribs, recesses or indentations on the parts of the instruments that are held by the surgeon during use.

### Method Embodiments

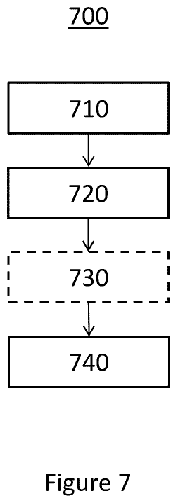

FIG. 7 is a flow diagram of embodiments of a method 700 for customizing an implant 300 adapted to be attached to an implant receiving surface 420 in a joint of a patient. In accordance with one or more embodiments, the method 700 comprises:

Step 710: obtaining a three-dimensional image representation of at least a part of the joint of the patient based on medical images generated using a medical imaging system 130.

Step 720: determining the shape and dimensions of a customized implant 300 adapted to be attached to the implant receiving surface 420, by designing a bone contacting surface 330 of the implant 300 to correspond to the implant receiving surface 420 and designing the implant 300 to comprise at least one implant peg 320 extending from the bone contacting surface 330.

Step 740: adding an osseointegrating structure 340, such as e.g. a lattice structure or a random lattice structure, to the bone contacting surface 330 of the implant 300 and at least a part of a surface area of the implant peg 320.

This enables the implant 300 to be properly secured to the implant receiving surface 420 in the bone, while still not being secured to the extent that it cannot be removed from the joint. In case of implant removal, it is usually desired that the osseointegration is limited to the area closest to the bone contacting surface 330 of the implant 300, to minimize unnecessary removal of bone, attached to the implant 300, from the joint.

In embodiments, the adding 740 of the osseointegrating structure 340 comprises adding the osseointegrating structure 340 along less than half of the length of the implant peg 320, for example along around one third of its length.

In embodiments of method, an osseointegrating structure is added in one or more of the following: in the form of a lattice structure with a depth in the range of 0.2-1.0 mm, for example a depth of 0.5 mm; and/or in the form of a lattice structure with a cell size in the range of 50-1500 micron, for example a cell size in the range of 100-1000 micron; and/or in the form of a lattice structure with a volume porosity in the range of 20-80%, for example a volume porosity in the range of 30-70%.

In embodiments, the determining 720 of the shape and dimensions of the customized implant 300 comprises designing the implant peg 320 to have a first diameter along most of its length, and a smaller diameter at the end (either with an edge where the first diameter suddenly changes into a second, smaller, diameter, or by the implant peg being tapered towards its end), and the adding 730 of the osseointegrating structure 340 comprises adding the osseointegrating structure 340 along less than two thirds of the length of the implant peg 320 having the first diameter, e.g. along around half of the length having the first diameter.

The method 700 may further comprise:

Step 730: generating the contour curvature of the articulating surface 310 using the three-dimensional image representation of the joint, to be based on the determined surface curvature of the cartilage and/or the bone in a predetermined area of the joint, to mimic the original, undamaged, articulating surface.

In embodiments, the generating 730 of the contour curvature of the articulating surface 310 comprises simulating a healthy articulating surface in the predetermined area, and designing the surface of the customized implant 300 to match the simulated healthy articulating surface.

Where applicable, various embodiments provided by the present disclosure can be implemented using hardware, software, or combinations of hardware and software. Also where applicable, the various hardware components and/or software components set forth herein can be combined into composite components comprising software, hardware, and/or both without departing from the claimed scope of the present disclosure. Where applicable, the various hardware components and/or software components set forth herein can be separated into sub-components comprising software, hardware, or both without departing from the claimed scope of the present disclosure. In addition, where applicable, it is contemplated that software components can be implemented as hardware components, and vice-versa. The method steps of one or more embodiments described herein may be performed automatically, by any suitable processing unit, or one or more steps may be performed manually. Where applicable, the ordering of various steps described herein can be changed, combined into composite steps, and/or separated into sub-steps to provide features described herein.

Software in accordance with the present disclosure, such as program code and/or data, can be stored in non-transitory form on one or more machine-readable mediums. It is also contemplated that software identified herein can be implemented using one or more general purpose or specific purpose computers and/or computer systems, networked and/or otherwise.

In embodiments, there are provided a computer program product comprising computer readable code configured to, when executed in a processor, perform any or all of the method steps described herein. In some embodiments, there are provided a non-transitory computer readable memory on which is stored computer readable and computer executable code configured to, when executed in a processor, perform any or all of the method steps described herein.

In one or more embodiments, there is provided a non-transitory machine-readable medium on which is stored machine-readable code which, when executed by a processor, controls the processor to perform the method of any or all of the method embodiments presented herein.

The foregoing disclosure is not intended to limit the present invention to the precise forms or particular fields of use disclosed. It is contemplated that various alternate embodiments and/or modifications to the present invention, whether explicitly described or implied herein, are possible in light of the disclosure. The figures and the detailed description specifically illustrate a metatarsal implant, but the disclosure applies to any kind of implant suitable for use in a joint of a patient. The joint may be e.g. a knee, an ankle, a hip, a toe, an elbow, a shoulder, a finger, or a wrist. The figures and the detailed description specifically illustrate an implant comprising one straight implant peg, but the implant peg does not have to be straight, and the implant may comprise more than one implant peg. Further, an implant peg in the shape of a screw may be used. A lattice structure or a random lattice structure are given as examples of osseointegrating structures, but other types of uneven surface structures may also be used. The surface structure may e.g. have a grid shape, or an entirely random shape, as long as there are a number of different levels of recesses, regular or irregular, in the surface structure. Accordingly, the scope of the invention is defined only by the claims.

1. An implant adapted to be attached to an implant receiving surface in a joint of a patient, the implant comprising a bone contacting surface and at least one implant peg extending from the bone contacting surface, wherein both the bone contacting surface and a part of a surface area of the implant peg comprises an osseointegrating structure, such as e.g. a lattice structure or a random lattice structure, wherein the surface area of the implant peg comprises said osseointegrating structure along less than half of the length of the implant peg.

2. The implant according to claim 1, wherein the surface area of the implant peg comprises the osseointegrating structure along around one third of the length of the implant peg.

3. The implant according to claim 1, wherein the lattice structure has a depth in the range of 0.2-1.0 mm, for example a depth of 0.5 mm.

4. The implant according to claim 1, wherein the lattice structure has a cell size in the range of 50-1500 micron, for example a cell size in the range of 100-1000 micron.

5. The implant according to claim 1, wherein the lattice structure has a volume porosity in the range of 20-80%, for example a volume porosity in the range of 30-70%.

6. The implant according to claim 1, wherein the articulating surface of the implant comprises titanium (Ti) or titanium alloy, titanium nitride (TiN) titanium niobium nitride (TiNbN), and/or a cobalt-chromium (CoCr) alloy.

7. The implant according to claim 1, comprising an articulating surface configured to correspond to the curvature of a simulated healthy articulating surface at a site of diseased cartilage and/or bone.

8. The surgical kit, comprising:
the implant according to claim 1; and
a bone processing tool guide comprising a contact surface configured to have a shape and contour that is designed to correspond to and to fit the actual surface contour in a predetermined area of a joint of a patient.

9. The surgical kit according to claim 8, further comprising an insert tool configured to be used for attaching the implant to the implant receiving surface, wherein the insert tool has an implant engaging portion that has a surface curvature that substantially corresponds to the surface curvature of the articulating surface of the implant.

10. A system for customizing an implant adapted to be attached to an implant receiving surface in a joint of a patient, the system comprising at least one processor configured to:
obtain a three-dimensional image representation of at least a part of the joint of the patient based on medical images generated using a medical imaging system;
determine the shape and dimensions of a customized implant adapted to be attached to the implant receiving surface by designing a bone contacting surface of the implant to correspond to the implant receiving surface, and designing the implant to comprise at least one implant peg extending from the bone contacting surface; and
add an osseointegrating structure, such as e.g. a lattice structure or a random lattice structure, to the bone contacting surface of the implant and to a surface area of the implant peg along less than half of the length of said implant peg.

11. The system according to claim 10, wherein the at least one processor is configured to add the osseointegrating structure along around one third of the length of the implant peg.

12. The system according to claim 10, wherein the at least one processor is configured to add an osseointegrating structure in the form of a lattice structure with a depth in the range of 0.2-1.0 mm, for example a depth of 0.5 mm.

13. The system according to claim 10, wherein the at least one processor is configured to add an osseointegrating structure in the form of a lattice structure with a cell size in the range of 50-1500 micron, for example a cell size in the range of 100-1000 micron.

14. The system according to claim 10, wherein the at least one processor is configured to add an osseointegrating structure in the form of a lattice structure with a volume porosity in the range of 20-80%, for example a volume porosity in the range of 30-70%.

15. The system according to claim 10, wherein the at least one processor is configured to determine the shape and dimensions of the customized implant using said three-dimensional image representation of the joint, by generating the contour curvature of the articulating surface based on the determined surface curvature of the cartilage and/or the bone in a predetermined area of the joint, to mimic the original, undamaged, articulating surface.

16. The system according to claim 15, wherein the at least one processor is configured to generate the contour curvature of the articulating surface by simulating a healthy articulating surface in the predetermined area, and designing the surface of the customized implant to match said simulated healthy articulating surface.

17. A method for customizing an implant adapted to be attached to an implant receiving surface in a joint of a patient, the method comprising:
obtaining a three-dimensional image representation of at least a part of the joint of the patient based on medical images generated using a medical imaging system;
determining the shape and dimensions of a customized implant adapted to be attached to the implant receiving surface, by designing a bone contacting surface of the implant to correspond to the implant receiving surface and designing the implant to comprise at least one implant peg extending from the bone contacting surface; and
adding an osseointegrating structure, such as e.g. a lattice structure or a random lattice structure, to the bone contacting surface of the implant and a surface area of the implant peg along less than half of the length of said implant peg.

18. The method according to claim 17, wherein the adding of the osseointegrating structure comprises adding the osseointegrating structure along around one third of the length of the implant peg.

19. The method according to claim 17, comprising adding an osseointegrating structure in the form of a lattice structure with a depth in the range of 0.2-1.0 mm, for example a depth of 0.5 mm.

20. The method according to claim 17, comprising adding an osseointegrating structure in the form of a lattice structure with a cell size in the range of 50-1500 micron, for example a cell size in the range of 100-1000 micron.

21. The method according to claim 17, comprising adding an osseointegrating structure in the form of a lattice structure with a volume porosity in the range of 20-80%, for example a volume porosity in the range of 30-70%.

22. The method according to claim 17, wherein the determining of the shape and dimensions of the customized implant uses said three-dimensional image representation of the joint, and comprises generating the contour curvature of the articulating surface based on the determined surface curvature of the cartilage and/or the bone in a predetermined area of the joint, to mimic the original, undamaged, articulating surface.

23. The method according to claim 22, wherein the generating of the contour curvature of the articulating surface comprises simulating a healthy articulating surface in the predetermined area, and designing the surface of the customized implant to match said simulated healthy articulating surface.
